(19) United States
(12) Patent Application Publication
(10) Pub. No.: US 2003/0148925 A1
(43) Pub. Date: Aug. 7, 2003

(54) Insulin formulation for inhalation

(76) Inventor: Jaap Kampinga, Groningen (NL)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/258,915

(22) PCT Filed: May 16, 2001
(86) PCT No.: PCT/GB01/02181

(30) Foreign Application Priority Data
May 16, 2000 (GB) 001807.5

(51) Int. Cl. A61K 38/28; A61K 31/724; A61K 31/7012

(52) U.S. Cl. 514/3; 514/53; 514/58

(57) ABSTRACT

A particulate composition comprising particulates having a mixture of 10 to 40% insulin and 90 to 60% saccharide is shown to be particularly suited for pulmonary delivery to a patient.

### FIELD OF THE INVENTION

This invention relates to a formulation of insulin suitable for systemic delivery via administration to the lung, and which has good stability.

### BACKGROUND OF THE INVENTION

There is now widespread interest in the formulation of therapeutic agents for inhalation. In particular, many efforts have been made to formulate suitable therapeutic agents as dry powders for delivery via inhalers.

Typically, the formulations are produced by drying the active agent in the presence of certain excipients, such as polysaccharides or citrate, to enhance stability during the drying process or in storage.

Insulin is a typical example of a therapeutic agent that can be administered to the lung, by inhalation. As a commercial product, insulin is generally provided in suspension or a solution of low concentration, as a hexamer complexed with zinc. Refrigeration is necessary, in order to maintain the stability of such a formulation. Crystalline Zn insulin is stable at neutral pH. The dry powder also requires refrigeration.

CA-A-2136704 discloses a product obtained by spray-drying a medicinal substance such as insulin (among many others) and a carrier. Example 4 discloses spray-drying a clear solution of human insulin, soya bean lecithin and lactose.

WO-A-9735562 again discloses spray-drying a solution of insulin and a polysaccharide. The aim of this combination is to achieve the preferred size range of spray-dried microparticles, for good lung deposition. In Examples 1 and 3, the insulin solution for spray-drying, prior to combination with polysaccharide, is prepared by dissolving zinc insulin in HCI, and then adding NaOH, to pH 7.2. The solutions for spray-drying respectively contain 25 and 6 mg/ml insulin and at least 5.5/7.2% NaCI, based on the combined weight of insulin plus salt.

WO-A-9524183 is directed primarily to a dry powder that comprises insulin and a carrier material, typically a saccharide, in the form of an amorphous powder of microparticles obtained by spray-drying. In addition, the Experimental section compares the properties of such microparticles with and without a saccharide excipient. The insulin solution for spray-drying is prepared by dissolving Zn-insulin in citrate buffer, at pH 6.7±0.3, to a solids content of 7.5 mg/ml. The powder is held in a container at 10% RH. For various reasons, this experiment cannot be reproduced: citrate is a buffer at pH 3.0-6.2, and not at pH 6.7; crystalline insulin will not dissolve in pH 6.2 citrate buffer before or after adjustment to pH 7.4 with NaOH; in any case, no alkali addition is specified.

Although there are various formulations of insulin disclosed in the prior art, there is still a recognised need for improved formulations, especially formulations which provide improved bioavailability when administered via the pulmonary route.

### SUMMARY OF THE INVENTION

The present invention is based on the surprising finding that particular ratios of insulin and saccharide show improved bioavailability, and are therefore very useful in pulmonary delivery.

According to a first aspect of the invention, a particulate composition for pulmonary delivery comprises particles having a mixture of 10 to 40% insulin and 90 to 60% saccharide.

In the most preferred formulation, the mixture is 20% insulin and 80% trehalose.

### DESCRIPTION OF THE DRAWINGS

The present invention is described with reference to the drawings, wherein:

FIG. 1 illustrates the whole blood glucose levels at various time points; and

FIG. 2 illustrates plasma insulin levels at various time points.

### DESCRIPTION OF THE INVENTION

The present invention provides new formulations of insulin and a suitable saccharide molecule for pulmonary delivery.

The formulations may be prepared by any suitable method known in the art, including, in particular, spray drying solutions of appropriate concentrations of the saccharide and insulin.

The insulin may be in any suitable form. For example, the insulin may be in the monomeric or hexameric form. Zinc insulin and other forms of insulin are also within the scope of the invention, e.g. insulin lispro, as are fragments of insulin that exert the appropriate therapeutic effect.

The saccharide component may be any suitable for pulmonary administration. The saccharide may be a monosaccharide, disaccharide or polysaccharide. In particular, the sugars lactose, sucrose and trehalose are preferred. Other saccharides including cyclodextrin may also be used.

Mixtures of saccharides may also be used to make up the saccharide component. This may be beneficial to prevent crystallisation on storage. In one embodiment, the saccharide component is a mixture of a polysaccharide and trehalose. In a further embodiment, the saccharide component is a mixture of pullulan and trehalose. Modified saccharides are also within the scope of the invention. For example, trehalose derivatives can be used as part of the particulate compositions. Other suitable saccharides will be apparent to the skilled person and are disclosed in International Patent Publication number WO-A-96/03978, the content of which is incorporated herein by reference. The preferred saccharides are the non-derivatised mono and disaccharides.

The saccharide should by physiologically acceptable. Depending on the method used to produce the particles, it may be desirable to use a saccharide with a high glass transition (Tg) temperature. If spray-drying is to be used, it is preferable to use a saccharide with a Tg above that of the inlet and outlet temperatures of the spray-drying apparatus, as otherwise, the saccharide may melt and stick to the inlet and outlet nozzles of the apparatus. It is also preferable to use a saccharide with a high Tg, as this may help to maintain stability of the particles on storage, particularly on storage at room temperature. A Tg of greater than 40° C. is therefore preferred, with a Tg of greater than 70° , being more preferred.

The particles are prepared so that residual moisture is minimised and the particles are in an amorphous form. It is preferable to have a residual moisture content of less than 5% (w/w). Determining the residual moisture can be carried out by known methods.

Although the preferred method for producing the particles is spray-drying, suitable alternative methods will be apparent to the skilled person. For example, freeze-drying may be used, with the resulting freeze-dried product being milled to produce the particles of the desired size for pulmonary delivery. A spray-freeze-drying process may also be used, as outlined in co-pending international patent application number PCT/GB01/00834. Other methods of making the formulation include, but are not limited to, air drying, vacuum drying, fluidised-bed drying, milling, co-precipitation and super-critical fluid processing.

The particles may be prepared either as solid solutions or solid dispersions. If a solid solution is required, the insulin may be prepared as in international patent application number PCT/GB99/02023. Alternatively, the insulin may be prepared as nanoparticles dispersed within the saccharide matrix.

In addition to the insulin and saccharide components, small quantities of additional components may be present. For example, minor amounts of salts or trace elements may be present.

The mixture of insulin to saccharide is 10 to 40% insulin to 90 to 60% saccharide. Preferably, the mixture is 15 to 30% insulin and 85 to 70% saccharide, more preferably 15 to 20% insulin and 85 to 80% saccharide. Most preferably the mixture is about 20% insulin and about 80% saccharide.

The particulate compositions are intended for pulmonary delivery to a patient. Devices suitable for delivery of the compositions are known, and will be apparent to the skilled person. The preferred delivery system is a passive dry powder inhaler (DPI), which relies entirely on the patient's inspiratory efforts to introduce the particles in a dry powder form into the lungs. However, alternative delivery devices may also be used. For example, active inhalers requiring a mechanism for delivering the powder to the patient may also be used. The particles may be formulated for delivery using a metered dose inhaler (MDI), which usually requires a high vapour pressure propellant to force the particles from the device.

The particles should preferably be 0.1 to 15 μm in diameter, more preferably 0.5 to 5 μm in diameter and most preferably 1 to 3 μm in diameter. The particles may be in a solid or porous form.

It will be appreciated that the particulate compositions are to be formulated in physiologically effective amounts. That is, when delivered in a unit dosage form, there should be a sufficient amount of the insulin to achieve the desired response. As the particles are intended primarily for delivery in dry powder inhalers, it will be appreciated that a unit dose comprises a predefined amount of particles delivered to a patient in one inspiratory effort. For guidance only, a single unit dose will be approximately 1 mg to 15 mg, preferably 5 mg to 10 mg of the particles. The delivery of the insulin particles is intended primarily for the treatment of diabetes.

The following example illustrates the invention.

### EXAMPLE

The objective of this study was to determine the bioavailability of 4 novel insulin dry powder formulations following administration by the inhalation route. Each test formulation was administered to 5 dogs and the plasma insulin and whole blood glucose levels were determined. Comparative bioavailability was assessed against a marketed insulin formulation (E) administered subcutaneously. Inhalation administration was undertaken via a surgically prepared tracheostome to allow direct entry to the bronchiopulmonary region of the lungs. The formulations tested are shown in Table 1.

TABLE 1
Test Material

| | |
|---|---|
| A. | (Zinc Insulin) |
| B. | (Insulin Without Zinc) |
| C. | (95% Zinc Insulin in Trehalose) |
| D. | (20% Zinc Insulin in Trehalose) |
| E. | (Humulin S) |

The four test materials coded A-D (for inhalation administration), were supplied as spray-dried powder formulations in glass vials, whilst formulation E (for subcutaneous administration) was supplied as a liquid. Formulations A-D were stored in the dark at ambient room temperature, whilst formulation E was stored at +4° C.

Formulation E (Humulin S) was supplied as a 100 IU/ml solution. The dose required for the pilot phase of the study was 1.5 IU/dog. Due to the small volumes of Humulin S required, this formulation was diluted with sterile water for injection to allow larger volumes of the correct dose level to be administered.

The study was conducted in 2 phases: a pilot phase followed by a main study.

Pilot Study

In order to provide baseline data, one dog (1M) was dosed subcutaneously (1.5IU) with a currently marketed insulin formulation (Humulin S) and the blood glucose and insulin levels determined over an approximately 4 h period.

Main Study

For the main study, 5 dogs (Animals 2-6) were used. Initially each dog received a subcutaneous dose of insulin (1.5 IU) to provide comparative plasma insulin and whole blood glucose levels. Following a minimum 2-3 day wash-out period, each dog was administered one of the 4 insulin formulations, in a randomised order, by direct inhalation exposure (7.5 IU) to an aerosol bolus delivered via a surgically prepared tracheostome. The remaining 3 insulin formulations were administered in a similar manner at approximately 2 day intervals. The tracheostome was surgically prepared, with the dogs under general anaesthesia, approximately 2 weeks before dosing.

The dosing regimen with estimated dosages is shown in Table 2.

The administered doses of insulin were derived by analytical determination by subtracting the amount of insulin retained in the dosing device from the total insulin loaded. The actual insulin units delivered are calculated based on the assumption that each milligram of insulin is equivalent to 28.6 units.

TABLE 2

| | | |
|---|---|---|
| Dog | Formulation | Insulin Dosed (units) |

Dose Session 2:

| | | |
|---|---|---|
| 2 | A | 10 |
| 3 | D | 13 |
| 4 | C | 3 |
| 5 | D | 3 |
| 6 | | no data |

Dose Session 3:

| | | |
|---|---|---|
| 2 | B | 5 |
| 3 | C | 6 |
| 4 | | no data |
| 5 | A | 8 |
| 6 | D | 11 |

Dose Session 4:

| | | |
|---|---|---|
| 2 | C | 7 |
| 3 | | no data |
| 4 | B | 6 |
| 5 | C | 5 |
| 6 | A | 7 |

Dose Session 5:

| | | |
|---|---|---|
| 2 | D | No data |
| 3 | B | 6 |
| 4 | A | 6 |
| 5 | B | 4 |
| 6 | C | 5 |

Dose Session 6:

| | | |
|---|---|---|
| 2 | | no data |
| 3 | A | 7 |
| 4 | D | 2 |
| 5 | | no data |
| 6 | B | 6 |

The animals were observed at least twice daily for signs of ill health or reaction to treatment. On the days of treatment, animals were observed continuously for reaction to treatment during dosing and at regular intervals up to approximately 4 h after dosing. Body weights were recorded once weekly whilst food consumption was recorded daily. Serial blood samples were obtained on each day of treatment to determine plasma insulin and whole blood glucose levels.

Results

Pilot Study

Following administration of Formulation F by the subcutaneous route (1.5IU/dog), an appropriate reduction was obtained for the whole blood glucose profile with a corresponding increase in plasma insulin levels.

Main Study

The values obtained appear to indicate a degree of variability in the estimated dose administered for all 4 inhaled formulations. Ranges recorded (units dosed) were—Formulation A: 6-10, Formulation B: 4-6, Formulation C: 3-7, and Formulation D: 2-13.

There were no adverse clinical signs observed on days of treatment or during the subsequent wash-out periods. Body weight and food consumption profiles were satisfactory over the course of the study. Bioavailability investigations revealed that all formulations produced a marked decrease in whole blood glucose levels and a correlating increase in insulin levels. This decrease in glucose and increase in insulin was most pronounced for Formulation D, i.e. 20% insulin and 80% trehalose.

Glucose Measurements

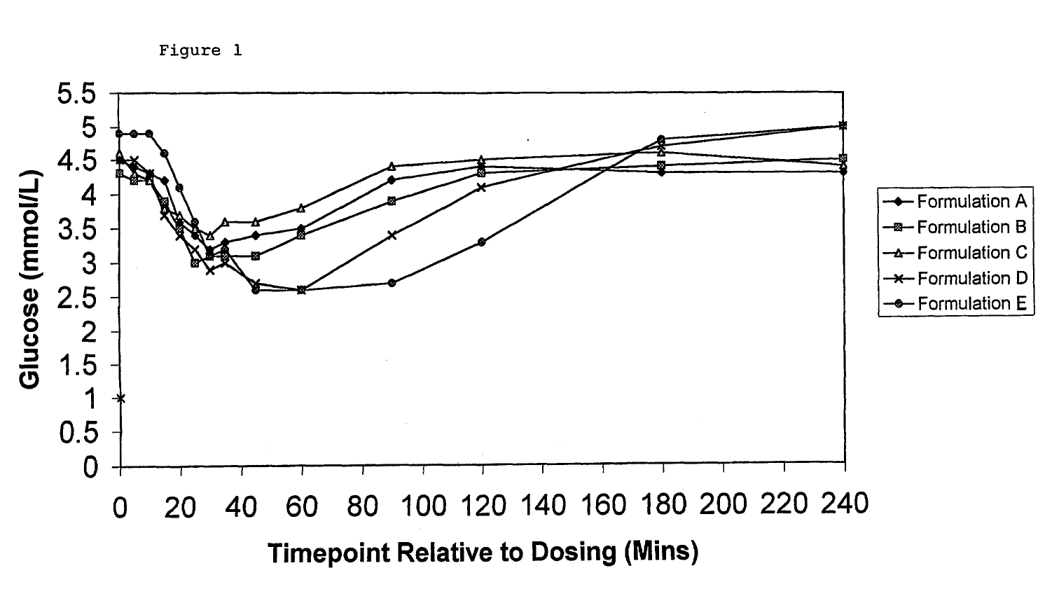

Mean glucose values per formulation are presented graphically in FIG. 1.

Glucose levels showed a steady decrease for all formulations with the lowest value occurring at about ±45 min after dosing. This decrease was most pronounced for Formulation D when compared against that obtained following administration of Formulation E by the subcutaneous route.

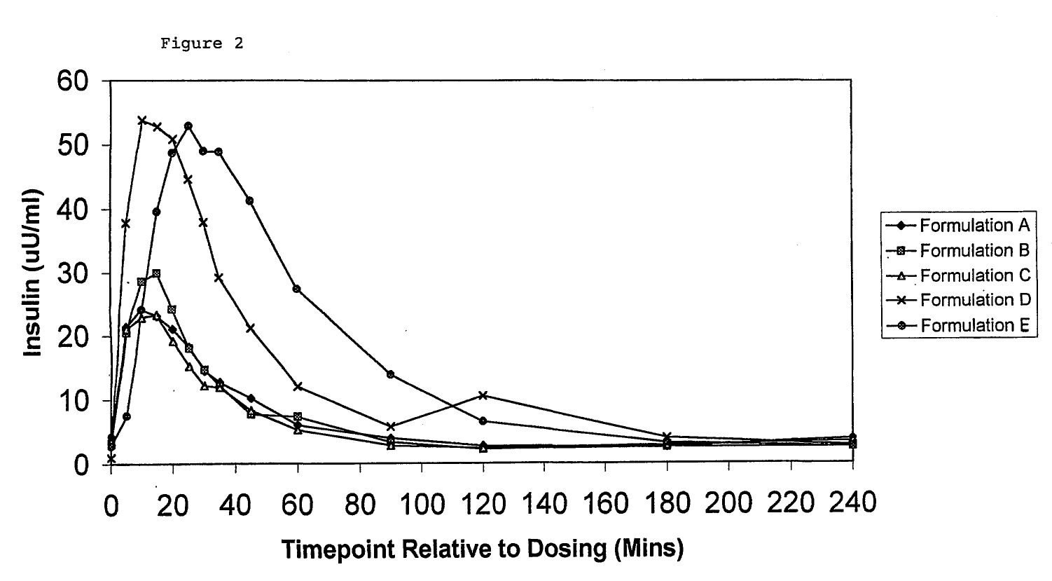

Mean insulin values per formulation are presented graphically in FIG. 2.

The decrease in glucose levels correlated with an increase in insulin levels for the animals treated with all formulations. The inhaled insulin formulations showed a rapid onset and decline of action when compared to the subcutaneous dose which produced a more sustained response. The increase was most pronounced for animals treated with Formulation D when compared against that obtained following administration of Formulation E. The peak increase occurred at about ±10-20 min after dosing for all formulations administered by the inhalation route. The inhaled formulations A and C produced comparable results and followed very similar response patterns.

A linear trapezoidal calculation of the area under the curve (AUC) was used to derive the values from the overall mean insulin blood concentration data. The values are presented in Table 3.

TABLE 3

| | AUC (uU.min/ml) | | |
|---|---|---|---|
| Formulation | Per Dose | Normalised* | (Relative %) |
| A | 657 | 123 | (4.2) |
| B | 773 | 234 | (8.0) |
| C | 625 | 188 | (6.4) |
| D | 2355 | 495 | (17.0) |
| E | 2916 | 2916 | (100) |

* = Relative to the subcutaneous dose (Formulation E) of 1.5 units

Following normalisation to the doses administered, it is apparent that Formulation D (20% Zinc Insulin in 80% Trehalose) provides the highest AUC, followed by Formulations B, C and A.

1. A particulate composition for pulmonary delivery, comprising particles having a mixture of 10 to 40% insulin and 90 to 60% of a saccharide.

2. A composition according to claim 1, wherein the insulin is zinc-free insulin.

3. A composition according to claim 1 or claim 2, wherein the Insulin is In monomeric form.

4. A composition according to any preceding claim, wherein the mixture is 15 to 30% insulin and 85 to 70% saccharide.

5. A composition according to any preceding claim, wherein the mixture is 15 to 20% insulin and 85 to 80% saccharide.

6. A composition according to any preceding claim, wherein the mixture is about 20% insulin and about 80% saccharide.

7. A composition according to any preceding claim, wherein the saccharide is trehalose.

8. A composition according to any of claims 1 to 6, wherein the saccharide is cyclodextrin.

9. A composition according to any preceding claim, wherein the particles are 0.1 to 15 μm in size.

10. A composition according to any preceding claim, wherein the particles are in amorphous form.

11. A particulate composition for pulmonary delivery, comprising particles having a mixture of about 20% insulin and about 80% trahalose.

12. A device for the delivery of a therapeutic agent via the pulmonary route, comprising a composition according to any preceding claim.
